(19) United States
(12) Patent Application Publication
(10) Pub. No.: US 2004/0167586 A1
(43) Pub. Date: Aug. 26, 2004

(54) Charge normalization for electrical stimulation of excitable tissue to facilitate the comparison of neural responses to behavioral measurements having different parameters

(76) Inventor: Edward H. Overstreet, Valencia, CA (US)

Correspondence Address: ADVANCED BIONICS CORPORATION, 25129 RYE CANYON ROAD, VALENCIA, CA, 91355, US

(21) Appl. No.: 10/783,291

(22) Filed: Feb. 19, 2004

Related U.S. Application Data
(60) Provisional application No. 60/449,041, filed on Feb. 21, 2003.

(51) Int. Cl. A61N 1/18

(52) U.S. Cl. 607/48

(57) ABSTRACT

A method is disclosed that allows the comparison of neural response measures made at one pulse duration to programs using pulse durations of arbitrary duration. The method converts both the neural response stimulation levels (i.e., the pulse amplitudes and pulse widths of the applied electrical stimuli at which a given neural response measure is obtained) and the program stimulation levels (i.e., the pulse amplitudes and pulse widths of the electrical stimuli provided by the operating program of the cochlear implant system) into units of charge/phase, or charge delivered per unit time.

The present application claims the benefit of U.S. Provisional Patent application Serial No. 60/449,041, filed Feb. 21, 2003, which application, including its Appendix A, is incorporated herein by reference in its entirety.

### BACKGROUND OF THE INVENTION

The present invention relates to neural stimulators, and more particularly cochlear implant systems. Even more particularly, the invention relates to a way of using charge normalization of the electrical stimuli that is applied to excitable tissue in order to facilitate the comparison of neural responses to behavioral measurements having different parameters.

Most neural stimulators apply a biphasic stimulation pulse to excitable tissue. A biphasic pulse is one that has a negative pulse followed immediately or a short time thereafter by a positive pulse; or, alternatively, a positive pulse followed by a negative pulse. When applying a biphasic pulse to excitable tissue, charge balance should be maintained. This means that the first pulse of the biphasic pair of pulses have the same absolute value charge that the second pulse has. Stated differently, the charge of the first pulse should be equal to, but of an opposite polarity, as the charge of the second pulse. See, e.g., U.S. Pat. No. 6,289,247, col. 10, lines 16-20. The U.S. Pat. No. 6,289,247 patent is incorporated herein by reference.

It is also common to refer to the “magnitude” of the applied stimulus, rather than the amplitude of the stimulus pulse, because it is recognized that the magnitude of an applied stimulus pulse is a function of both the amplitude and the pulse width of the stimulus pulse. Thus, for example, a stimulus current pulse having an amplitude of, e.g., 1 milliamp (ma), and a pulse width of 1 millisecond (ms), is viewed as having the same magnitude as a stimulus current pulse having an amplitude of 2 ma and a pulse width of 0.5 ms. This is because the total charge in the respective pulses (pulse amplitude times pulse width) is the same. This concept of adjusting pulse amplitude and/or pulse width to achieve a desired “magnitude” of the stimulus pulse is also taught in PCT International Publication No. WO 01/43818 A1, published 21 Jun. 2001, incorporated herein by reference.

With modern cochlear implant systems, it is possible to generate programs that use electrical stimulation pulses having pulse durations (widths) ranging from a few microseconds (μs) to a few hundred microseconds. In cases where the patient may be extremely young or difficult to program due to other issues, the recording of neural responses is often done to better understand where the “ball-park” programed stimulus pulse amplitudes are likely to be. The problem is that it is common for the pulse widths of the stimuli used to elicit the neural response to be different than the pulse widths that are applied to the patient by the operating program of the cochlear implant system during its everyday use. It is not practical or time efficient to record the neural responses at all possible program pulse widths. Therefore, what is needed is a way to relate the neural responses obtained at one pulse duration (or pulse width) to programmed amplitudes (levels) of arbitrary pulse durations (or pulse widths). (Note: as used herein the term “pulse duration” is viewed as the same as “pulse width”; and “level” is normally viewed as the same as “amplitude”, but the context may indicate that “level” sometimes refers to “magnitude”.)

Representative background art related to the invention may be found, e.g., in the patents and publications referenced above, and in U.S. Pat. Nos. 6,4515,185; 6,295,467; 6,219,580; 6,205,360; 6,195,585; and 5,626,629, each of which is incorporated herein by reference.

### SUMMARY OF THE INVENTION

The present invention addresses the above and other needs by providing a method that relates neural and behavioral measures obtained at arbitrary pulse durations using a charge-based model with the allowance of a compensation factor for rate differences based upon temporal integration. Stated differently, the invention allows the comparison of neural response measures made at one pulse duration to programs using pulse durations of arbitrary duration. This is accomplished by converting both the neural response stimulation levels (i.e., the pulse amplitudes and pulse widths of the applied electrical stimuli at which a given neural response measure is obtained) and the program stimulation levels (i.e., the pulse amplitudes and pulse widths of the electrical stimuli provided by the operating program of the cochlear implant system) into units of charge/phase, or charge delivered per unit time.

### BRIEF DESCRIPTION OF THE DRAWINGS

The features and advantages of the present invention will be more apparent from the following more particular description thereof, presented in conjunction with the following drawings wherein:

FIG. 1A shows a simplified block diagram of a neural stimulation system;

FIG. 1B illustrates a biphasic pulse, and is used to define the terms amplitude, pulse width, and frequency;

FIG. 1C shows representative neural response waveforms;

FIG. 2 illustrates the Input/Output function of neural responses, and is used to define the parameter tNRI;

FIG. 3 illustrates the concept of constant charge pulses;

FIG. 4 presents data from a patient stimulated with constant charge pulses of various pulse widths;

FIG. 5 presents data from 18 different patients showing the relationship between the threshold NRI (one type of measured neural response) and the program M level after charge normalization.

In addition to the above drawings, reference is also made to Appendix A of U.S. Provisional Application Serial No. 60/449,041, filed Feb. 21, 2003, previously incorporated herein by reference, the benefit of which is claimed by this patent application. Appendix A of said provisional application teaches the relationships between the evoked compound action potential (ECAP) and HiResolution program settings in patients using the CII Bionic Ear cochlear implant system. Note that the CII Bionic Ear cochlear implant system is manufactured by Advanced Bionics Corporation, of Sylmar, Calif. The HiResolution (or “HiRes”) program settings of the CII Bionic Ear refer to a particular processing strategy used by the sound processor of that system.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

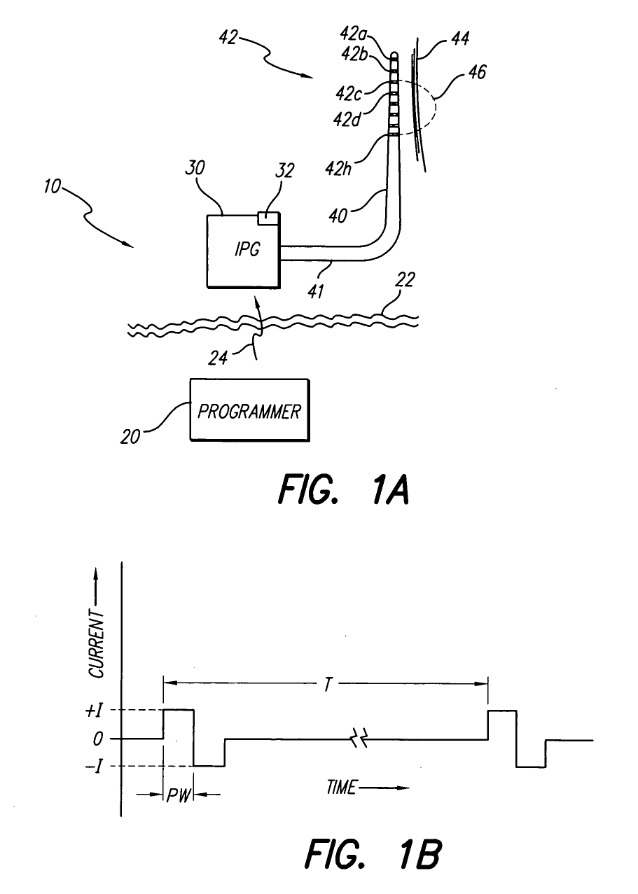

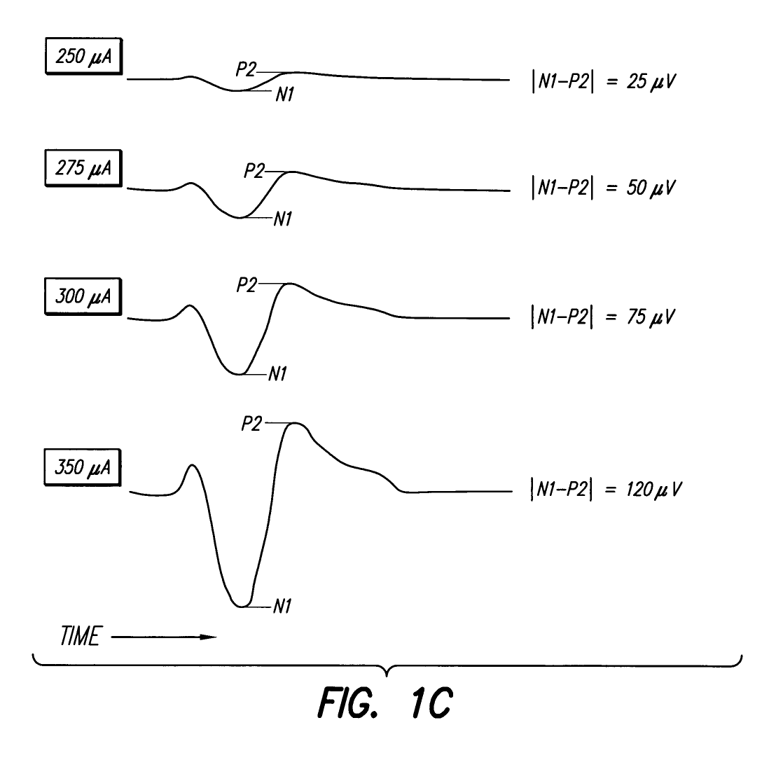

Turning first to FIG. 1A, there is shown a simplified block diagram of a neural stimulation system 10. The stimulation system 10 includes an implantable pulse generator (IPG) 30 connected to an electrode array 40. The electrode array 40 comprises a flexible carrier lead body 41, typically made from silicone rubber, or some other form of Silastic® material, on which individual spaced-apart electrode contacts 42a, 42b, 42c, . . . are located near a distal end. In a cochlear stimulation system (which represents one type of neural stimulation system), the electrode array 40 is adapted to be inserted into the cochlear of a patient so that electrical stimuli can be applied through the electrode contacts directly to the auditory nerve.

In FIG. 1A, there are eight electrode contacts illustrated, contacts 42a, 42b, 42c, . . . 42h. However, this is only exemplary. Any number of electrode contacts may be used. Typically, for a cochlear stimulation system, sixteen or more electrode contacts are used. Any number of electrode contacts 42 may be used depending upon the application. At least two electrode contacts, one of which may be included on the case of the IPG 30, must be used to produce a stimulation.

The electrode array 40 which is shown in FIG. 1 is referred to as an “in-line” electrode array because all of the electrode contacts 42 are spaced-apart and are in line with each other along the body of the lead carrier body 41. An in-line electrode array is typically used for a cochlear stimulation system. However, different types of electrode arrays may be used, e.g., various forms of paddle arrays, or combinations of in-line arrays, for other types of neural stimulation systems, e.g., a spinal cord stimulation system.

Each electrode contact 42a, 42b, 42c, . . . 42h of the electrode array 40 is connected by way of a wire (not shown in FIG. 1A), or other electrical conductor, embedded within the lead body to electronic pulse generation circuitry housed within the IPG 30. It is through these wires or conductors that an electrical stimulation pulse is delivered to the electrode contacts 42.

It is the purpose of the electrode array 40 to place the multiplicity of electrode contacts 42 near the body tissue that is to be stimulated. For example, in FIG. 1, a nerve fiber 44, or other similar tissue (e.g., muscle or nerve tissue), is shown as being positioned alongside the electrode array contacts 42. When one of the electrode contacts, e.g., contact 42c, is selected as an anode, and another of the electrode contacts, e.g., contact 42h, is selected as a cathode, then a stimulation current pulse, generated by the IPG 30, may flow along a current path, represented by the dotted line 46, that flows through the body tissue and the nerve fiber 44, thereby stimulating the nerves or other body tissue that come in contact with the current path so long as the current flowing through such path is of a sufficient magnitude to evoke a stimulus response. More than one electrode contact may be selected as the anode, and/or as the cathode, in such stimulation. Additionally, the IPG 30 may have a reference electrode 32 located, e.g., on its case so that a stimulation current path may be set up between one or more of the electrodes 42 included within the electrode array 40 and the reference electrode 32. Such electrode configuration, wherein one of the electrode contacts is the reference electrode 32, and one of the electrodes 42 of the electrode array 40 is used as the other electrode, is referred to as monopolar stimulation. An electrode configuration where two of the electrodes 42 are used as the electrodes, one as the anode and one as the cathode, is referred to as bipolar stimulation. An electrode configuration where three of the electrodes 42 are used as the electrodes, one or two as the anode, and one or two as the cathode, is referred to as tripolar stimulation. Similarly, an electrode configuration where four or more of the electrodes 42 are used as the electrodes, at least one as the anode and at least one as the cathode, is referred to as multipolar stimulation.

Thus, it is seen that the multiplicity of electrode contacts 42 located on the electrode array 40, with or without the reference electrode 32 located on the case of the IPG 30, provide a large number of possible electrode combinations that could be used when applying stimulation pulses to the body tissue. When all of these possible electrode combinations are further combined with all of the possible parameter changes that could be made to the applied stimulus pulse(s), it is evident that the number of combinations that need to be tested in order to determine an optimum safe and efficacious combination for use by the patient is almost unmanageable. One purpose of the present invention to provide a way to quickly and safely identify which electrode combinations, and which stimulus parameters, are most efficacious for a given patient.

The IPG 30 is controlled by, e.g., programmed by, an external programmer 20. The programmer 20, or a component of the programmer 20 (e.g., a programming head) is typically placed near the skin 22 of the patient in an area close to the location where the IPG 30 is implanted. Then, a suitable transcutaneous communications link, represented in FIG. 1A by the wavy arrow 24, is established between the programmer 20 and the IPG 30. Such link 24 may take many forms, any of which is suitable for purposes of the present invention. Exemplary transcutaneous links 24 may be realized, e.g., through inductive coupling, RF transmission, magnetic coupling, optical coupling, and the like.

Once the transcutaneous link 24 has been established, the programmer 20 downloads whatever programming data the IPG 30 needs in order to perform its function of generating stimulation pulses. Such data typically includes the stimulus parameters, e.g., pulse width, pulse amplitude, and pulse rate (defined below in connection with FIG. 1B), and the individual electrode contacts 42 through which the electrical stimulus is to be applied to the body tissue. Such data may also include new or updated operating programs to be used by the IPG as it carries out its function of generating stimulus pulses. In many embodiments of the invention, the IPG 30 may also send status and/or other data back to the programmer 20 through a back telemetry link. When used, the back telemetry link may be the same as, or different from, the forward transcutaneous link 24.

FIG. 1B illustrates a typical stimulation pulse waveform that might be used with a neural stimulator. The stimulation pulses shown in the waveform of FIG. 1B are biphasic pulses, having a first pulse of one polarity followed immediately by a pulse of the same magnitude but opposite polarity. A biphasic pulse offers the advantage of balancing the electrical charge that flows through the body tissue, which is generally considered to be an important consideration. Other pulse waveforms may also be used to provide a charge balanced condition, as is known in the art.

The waveform shown in FIG. 1B is useful, for purposes of the present invention, because it defines the common stimulus parameters that are used to define the electrical stimuli generated by a neural stimulator, such as a cochlear implant device. These stimulus parameters include, e.g., pulse width (PW), pulse amplitude, and pulse rate. As seen in FIG. 1B, the pulse width is the duration or width, measured in units of time (such as microseconds or milliseconds), of the individual pulses that make up the biphasic pulses.

Still referring to FIG. 1B, the pulse amplitude is the magnitude of the peak current in the pulse, measured in units of electrical current, e.g., milliamperes (mA). In FIG. 1B, the amplitude is shown as “I”. One pulse of the biphasic pulse has an amplitude of +I, and the other pulse has a magnitude of −I. It should be noted that FIG. 1B shows a current stimulation pulse. It is also possible to stimulate with a voltage stimulation pulse, in which case the amplitude would be expressed in units of volts, and the electrical current that actually is applied to the tissue would be a function of the impedance of the electrode/tissue interface and the tissue itself.

As also seen in FIG. 1B, the stimulation rate is determined by the inverse of the period T between recurring pulses. That is, the rate, expressed in units of pulses per second, or Hz, is equal to 1/T, where T is the time increment between corresponding parts, e.g., leading edges, of the biphasic (or other) pulses. The pulse rate, depending upon the application, may also be expressed in units of pulses per minute (PPM). The ratio of the pulse width (PW) to the time T (the increment between the pulses) defines the duty cycle.

In accordance with the present invention, once a neural stimulation system with a suitable electrode array has been implanted within a patient, a programmer, or equivalent device, is used to determine appropriate threshold levels, e.g., the perception threshold levels and the maximum tolerable threshold levels, associated with electrical stimuli applied through a selected set or group of electrode contacts on the electrode array. Such threshold data has heretofore required obtaining subjective feedback from the patient as stimuli of various magnitudes are applied through various electrode combinations. However, when the patients are very young, or other patient-communication abilities are impaired, such data may also include an objectively-measured neural response. For such patients, using such objectively-measured neural response makes the programming of the neural stimulator much more effective, with less trial-and-error work and guess-work. Unfortunately, obtaining the needed neural response for a given electrode configuration can be very time consuming, especially when a different electrical stimuli must be applied to obtain an appropriate neural response. A key feature of the present invention is that a set of neural responses collected at a single pulse-width can be compared to, or used to generate, programs of arbitrary pulse width without requiring the re-measurement of neural responses.

FIG. 1C depicts some typical neural response waveforms that occur when a stimulus is applied. The top waveform in FIG. 1C is the neural response that occurs when a 250 microamp (μA) pulse is applied. The second waveform from the top is the neural response that occurs when a 275 μA pulse is applied. The third waveform from the top is the neural response that occurs when a 300 μA pulse is applied. The bottom waveform is the neural response that occurs when a 350 μA waveform is applied. Thus, it is seen that when the amplitude of the applied stimulation pulse is increased, the size of the neural response grows as measured by the increasing amplitude of the peak-to-peak response. In the waveforms shown in FIG. 1C, the first negative peak is called N1 and the following positive peak is called P2. The N1-P2 difference is what is meant by the term neural response amplitude.

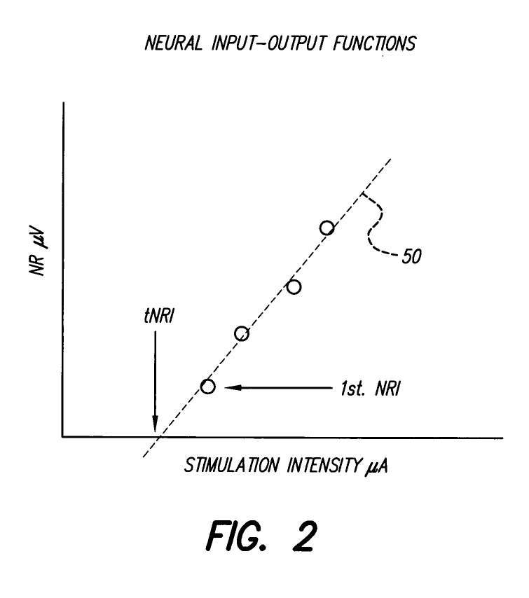

FIG. 2 shows a graph that depicts the relationship between the applied stimulus pulse, or stimulation intensity (the “input”, and the resulting neural response (the “output”). The stimulation intensity input is plotted on the x-axis (in μA), and the neural response output is plotted on the y-axis (in microvolts, or μV).

When comparing the neural response to the programs running on a cochlear implant, or other neural stimulator, the two most relevant measures are the 1st NRI ((Neural Response Imaging), which may correspond to a visual detection threshold, and the tNRI, which is the data-derived X-intercept extrapolation of the linear-regression line 50 for the data points, as shown in FIG. 2. (It should be noted that other measures of thresholds and other neural response parameters, other than the tNRI and the 1st NRI may also be used with the method of the present invention.) Previous research has shown that such threshold parameters, e.g., the tNRI and the 1st NRI, of the neural response typically fall between the T and M levels of the patient's program. (The “T” and “M” levels are used by the programmer 20 to set the stimulation intensity of the pulses that are delivered to the target tissue through the electrode array. The “T” level represents the minimum threshold level at which a patient first senses a stimulus. The “M” level represents the maximum threshold level at which the stimulus is still at a comfortable level, i.e., not so loud as to be painful. Thus, the programmer generally is programmed to deliver a stimulus that is greater than the “T” level, and less than the “M” level. For a further explanation of “T” and “M” levels, see, e.g., U.S. Pat. Nos. 5,626,629 and 6,289,247.) Thus, the 1st NRI and tNRI help to provide a “ball-park” target for stimulation levels when behavioral measures are difficult or impossible to obtain (as is often the case with young patients, or elderly patients).

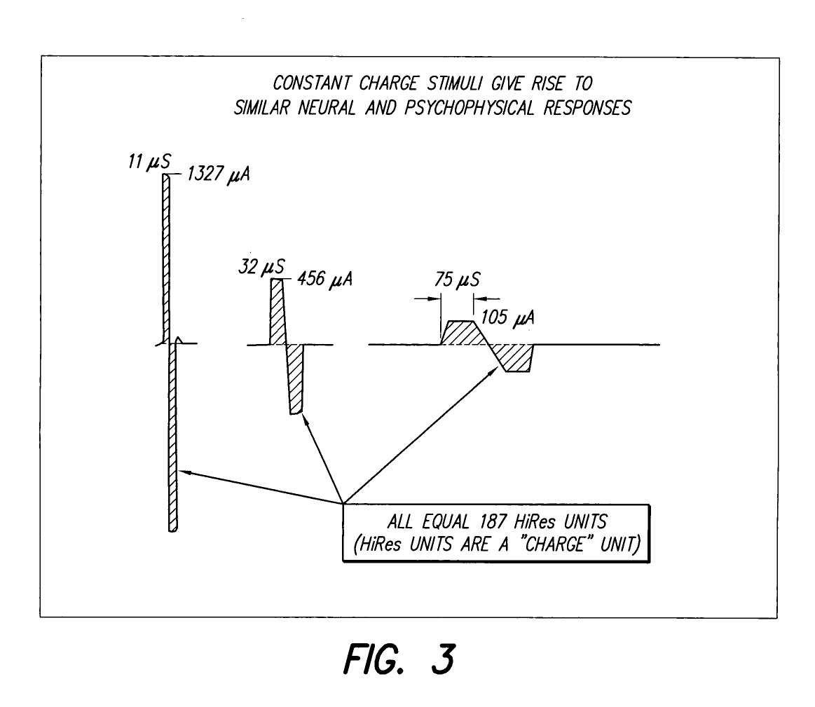

Turning next to FIG. 3, there are shown pulses of various pulse durations having the amplitude scaled to give a constant area of the pulse. This teaches that within an acceptable tolerance of error, normalizing stimulus parameters to “charge”—where charge may be expressed as the area of the pulse, i.e., the pulse amplitude times the pulse width—allows the comparison of neural responses obtained at one pulse width or pulse amplitude to programmed levels having different pulse parameters (pulse amplitudes and/or pulse widths).

Thus, it is seen that the present invention involves using “charge” as a normalized stimulus parameter, rather than pulse amplitude or pulse width, and then the “charge” levels corresponding to the 1st NRI and tNRI responses (or other suitable neural response thresholds) may be readily correlated to programmed levels that correspond to the same “charge”. This allows the programmed levels to be in the “ball-park”, i.e., less than the M levels and greater than the “T” levels, without having to actually subjectively measure the “M” and “T” levels (which subjective measurements of the “M” and “T” levels could be difficult, if not impossible, for certain types of patients, e.g., young children and elderly persons who have a difficult time communicating).

It is further seen that if a multiplicity of data points are taken, each with a different stimulation intensity, resulting in a plot of the data on a graph as shown in FIG. 2, that the charge associated with the tNRI point may be determined by, e.g., multiplying the pulse amplitude by the pulse width used to take the data points. In general, when a multiplicity of data points are taken to determine a tNRI data point as shown in FIG. 2, all of the data points are taken using stimuli having the same pulse width but different pulse amplitudes. Alternatively, all of the data points may be taken using stimuli having the same pulse amplitude but varying pulse widths.

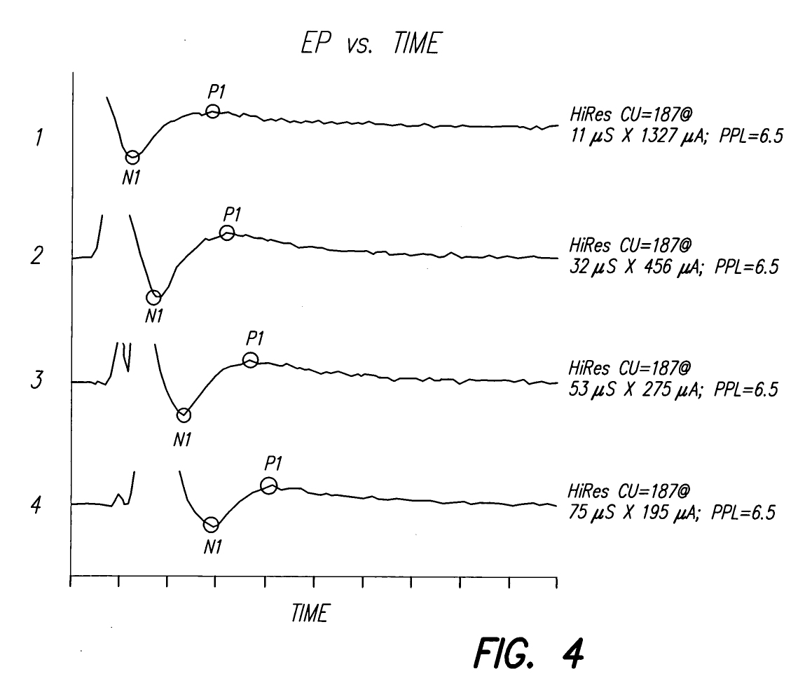

FIG. 4 illustrates use of a normalized “charge” parameter. In FIG. 4, the neural response time waveforms for four different stimulus pulse widths are shown, each having 187 “charge” units. In FIG. 4, the top waveform is the neural response corresponding to an applied stimulation pulse having a pulse width of 11 μs and a pulse amplitude of 1327 μA. The “charge” for this pulse is 11 μs×1327 μA=14597, which corresponds to a psychophysical level (PPL) of 6.5 on a loudness scale of 1-10. The second waveform from the top in FIG. 4 is the neural response corresponding to an applied stimulation pulse having a pulse width of 32 μs and a pulse amplitude of 456 μA. The “charge” for this second pulse is 32 μs×456 μA=14592, which also corresponds to a PPL of about 6.5. The third waveform from the top in FIG. 4 is the neural response corresponding to an applied stimulation pulse having a pulse width of 53 μs and a pulse amplitude of 275 μA. The “charge” for this third pulse is 53 μs×275 μA=14575, which also corresponds to a PPL of about 6.5. Finally, the bottom or fourth waveform in FIG. 4 is the neural response corresponding to an applied stimulation pulse having a pulse width of 75 μs and a pulse amplitude of 195 μA. The “charge” for this fourth pulse is thus 75 μs×195 μA=14625, again corresponding to a PPL of about 6.5. Thus, all of the neural response waveforms shown in FIG. 4 have approximately the same “charge” associated therewith.

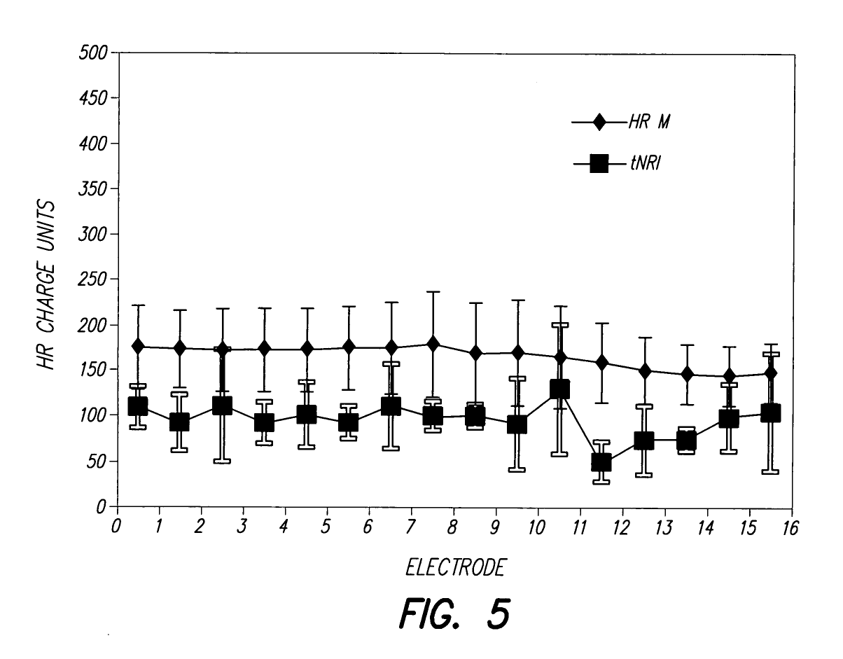

To prove the concept of the invention, data from 18 different subjects was obtained to show the relationship between the tNRI and the program “M” level after charge normalization. The data are shown in FIG. 5.

FIG. 5 shows the average “M” levels for the 18 patients on each of sixteen electrodes that have program pulse durations ranging from 11 to 53 μs. Also shown are the average tNRI values collected at a 32 μs pulse width as a function of the electrode on which the 32 μs pulse is applied. All stimulus parameters are plotted in HiRes charge units, where a HiRes charge unit is defined as: (pulse amplitude in μA)×(pulse width in μs)×k, where k is a scaling factor equal to 0.0128447.

Advantageously, using the method of the present invention, a clinician does not need to re-measure neural response values each time a pulse-duration change is made to the patient's program. Rather, using the method of the invention, one can use a predictive value of the neural responses collected at a single pulse duration for programs of arbitrary pulse duration.

The invention also extends to an implantable neural stimulation system, e.g., a cochlear implant system, that includes an implantable pulse generator, an electrode array, and means for programming and controlling the implantable pulse generator so that: (a) a threshold neural response elicited from application of at least one stimulus having a known amplitude and pulse width is determined; (b) the charge associated with the stimulus that produced the threshold neural response is determined; and (c) the program levels of the stimuli applied by the neural stimulation system during its operation are set to stimuli having approximately the same charge as the charge of the stimulus that produced the threshold neural response. Such implantable neural stimulation system may be realized using a hardware and software/firmware platform as described above in connection with 1A, and also as described, e.g., in U.S. Pat. Nos. 6219580; 6487453 and 6516227, which patents are incorporated herein by reference.

While the examples presented herein use a charge time window of one pulse, it is to be understood that larger windows could also be used to further increase accuracy and to compensate for stimulation rate.

A modern cochlear implant system, e.g., the CII Bionic Ear™ manufactured by Advanced Bionics Corporation of Sylmar, Calif., is equipped with a differential amplifier that allows the clinician to record electrically evoked compound action potentials (ECAP). The ECAP is a type of neural response. The utility of being able to measure ECAP for fitting cochlear implants implementing conventional low-rate sound processing strategies has been studied extensively, and the relationship between perceptual loudness and neural response magnitude is not straightforward. At present, it is unknown how HiResolution™ sound processing, designed to deliver high-rate stimuli in order to desynchronize neural responses to the carrier, will relate to ECAP measurements generated by single pulse stimuli that produce high neural synchrony.

Because HiResolution™ programs are created using an adaptive protocol that customizes individual-patient program parameters, in most cases, users of the CII Bionic Ear cochlear implant system, or other modern cochlear implant systems, will have HiResolution™ stimulating pulse widths that are different from the pulse widths used to elicit the ECAP. As a way of exploring the relationships between these stimuli, the ECAP and loudness percepts elicited by constant charge pulses ranging in pulse width from 11 to 75 microseconds was studied. As a first approximation, the data (see FIG. 5) indicate that constant-charge pulses give rise to similar perceptions of loudness and similar ECAP magnitudes. Thus, normalizing to constant charge provides a way to compare various HiResolution™ program levels to the ECAP. More information related to this study is found in Appendix A of the provisional patent application referenced in paragraph [0001] of this application.

While the invention herein disclosed has been described by means of specific embodiments and applications thereof, numerous modifications and variations could be made thereto by those skilled in the art without departing from the scope of the invention set forth in the claims.

What is claimed is:

1. A method for stimulating using a neural stimulation implant system comprising:
determining a threshold neural response elicited from application of at least one stimulus having a known amplitude and pulse width;
determining the charge associated with the stimulus that produced the threshold neural response; and
setting the program levels of the stimuli applied by the implant system during its operation to stimuli having approximately the same charge as the charge of the stimulus that produced the threshold neural response.

2. The method of claim 1 wherein determining a threshold neural response comprises
applying a multiplicity of stimuli, each applied stimuli having a different intensity level associated therewith,
measuring a corresponding neural response associated with application of each one of the multiplicity of stimuli,
defining a data point for each applied stimulus and its corresponding neural response, thereby defining a multiplicity of data points, and
analyzing the multiplicity of data points to determine the threshold neural response.

3. The method of claim 2 wherein measuring a corresponding neural response associated with application of each one of the multiplicity of stimuli comprises measuring a first peak N1 of one polarity of a neural response, and measuring a second peak P1 of an opposite polarity of the neural response, and determining the measured neural response to be the N1-P1 peak-to-peak difference.

4. The method of claim 3 wherein analyzing the multiplicity of data points to determine the threshold neural response comprises:
plotting the multiplicity of data points on a graph, wherein the intensity level of the applied stimulus comprises a first axis of said graph, and the measured neural response associated with the applied stimulus comprises a second axis of said graph;
fitting a data line to the multiplicity of data points plotted on the graph;
determining an intersection point at which an extrapolation of the data line intersects with the first axis of the graph; and
setting the threshold neural response as the intersection point.

5. The method of claim 4 wherein fitting a data line to the multiplicity of data points comprises fitting a straight line to the multiplicity of data points.

6. The method of claim 2 wherein applying a multiplicity of stimuli, each applied stimuli having a different intensity level associated therewith, comprises applying stimuli having different pulse amplitudes and a fixed pulse width, and wherein determining the charge associated with the stimulus that produced the threshold neural response comprises multiplying the amplitude of the threshold neural response by the fixed pulse width.

7. The method of claim 2 wherein applying a multiplicity of stimuli, each applied stimuli having a different intensity level associated therewith, comprises applying stimuli having different pulse widths and a fixed pulse amplitude, and wherein determining the charge associated with the stimulus that produced the threshold neural response comprises multiplying the pulse width of the threshold neural response by the fixed pulse amplitude.

8. A method for fitting a cochlear implant system comprising:
measuring a plurality of neural responses elicited from a corresponding plurality of applied stimuli, each applied stimuli having a different intensity level associated therewith;
determining a tNRI value from the measured plurality of neural responses;
using the tNRI value to guide the selection of an initial operating point for the cochlear implant system by:
determining the charge level associated with the tNRI value, where charge level is defined as pulse width X pulse amplitude, and
setting the programmed levels used by the cochlear implant system to stimuli having approximately the same charge as the charge associated with the tNRI value.

9. The method for fitting of claim 8 wherein determining a tNRI value comprises:
plotting the measured neural responses as data points on a graph having the applied stimuli as a first axis and the measured neural response as a second axis,
making a straight line approximation of the plotted data points, and
determining the tNRI value as the value corresponding to the intersection of the straight line approximation of the plotted data points with the first axis.

10. A neural stimulation system comprising an implantable pulse generator, an electrode array attached to the pulse generator, and control circuitry for controlling operation of the implantable pulse generator, wherein the control circuitry includes:
means for determining a threshold neural response elicited from application of at least one stimulus having a known amplitude and pulse width;
means for determining the charge associated with the stimulus that produced the threshold neural response; and
means for setting the program levels of the stimuli applied by the implant system during its operation to stimuli having approximately the same charge as the charge of the stimulus that produced the threshold neural response.

11. The neural stimulation system of claim 10 wherein the means for determining a threshold neural response comprises
means for applying a multiplicity of stimuli, each applied stimuli having a different intensity level associated therewith,
means for measuring a corresponding neural response associated with application of each one of the multiplicity of stimuli,
means for defining a data point for each applied stimulus and its corresponding neural response, thereby defining a multiplicity of data points, and
means for analyzing the multiplicity of data points to determine the threshold neural response.

12. The neural stimulation system of claim 11 wherein the means for measuring a corresponding neural response associated with application of each one of the multiplicity of stimuli comprises means for measuring a first peak N1 of one polarity of a neural response, and means for measuring a second peak P1 of an opposite polarity of the neural response, and means for determining the measured neural response to be the N1-P1 peak-to-peak difference.

13. The neural stimulation system of claim 11 wherein the means for analyzing the multiplicity of data points to determine the threshold neural response comprises:
means for plotting the multiplicity of data points on a graph, wherein the intensity level of the applied stimulus comprises a first axis of said graph, and the measured neural response associated with the applied stimulus comprises a second axis of said graph;
means for fitting a data line to the multiplicity of data points plotted on the graph;
means for determining an intersection point at which an extrapolation of the data line intersects with the first axis of the graph; and
means for setting the threshold neural response as the intersection point.

14. The neural stimulation system of claim 13 wherein the means for fitting a data line to the multiplicity of data points comprises means for applying a linear-regression line to the multiplicity of data points.

15. The neural stimulation system of claim 10 wherein the neural stimulation system comprises an implantable cochlear stimulation system.

16. The neural stimulation system of claim 10 wherein the neural stimulation system comprises a spinal cord stimulation system.
